(19) United States
(12) Patent Application Publication
(10) Pub. No.: US 2008/0108100 A1
(43) Pub. Date: May 8, 2008

(54) Method of determining lymph node metastasis

(75) Inventors: Naomi Zurggil, Herzlia (IL); Mordechai Deutsch, Doar-Na Lev HaSharon (IL)

Correspondence Address: Martin D. Moynihan;PRTSI, Inc., P.O. Box 16446, Arlington, VA, 22215, US

(73) Assignee: Seng Enterprises Ltd., Larnaca (CY)

(21) Appl. No.: 11/898,294

(22) Filed: Sep. 11, 2007

Related U.S. Application Data
(60) Provisional application No. 60/843,405, filed on Sep. 11, 2006.

(51) Int. Cl. C12Q 1/20

(52) U.S. Cl. 435/19; 435/29

(57) ABSTRACT

A method of determining lymph node metastasis in a subject is disclosed, comprising determining at least one structural or functional parameter of a lymph node cell of a lymph node tissue of the subject, wherein an alteration in the at least one structural or functional parameter with respect to a lymph node cell of a healthy lymph node tissue is indicative of lymph node metastasis. Kits and articles of manufacture are also disclosed for determining lymph node metastasis in a subject as well as methods of diagnosing cancer in general.

### RELATED APPLICATION

This application claims the benefit of U.S. Provisional Patent Application No. 60/843,405 filed Sep. 11, 2006, the contents of which are herein incorporated by reference.

### FIELD AND BACKGROUND OF THE INVENTION

The present invention relates to methods of determining lymph node metastasis and methods of treating same.

A major challenge of cancer treatment is to target specific therapies to distinct tumor types in order to maximize efficacy and minimize toxicity. A related challenge lies in the attempt to provide accurate diagnostic, prognostic, and predictive information. At present, tumors are described with the tumor-node-metastasis (TNM) system. This system, which uses the size of the tumor, the presence or absence of tumor in regional lymph nodes, and the presence or absence of distant metastases, to assign a stage to the tumor is described in the American Joint Committee on Cancer: AJCC Cancer Staging Manual. Philadelphia, Pa.: Lippincott-Raven Publishers, 5th ed., 1997, pp 171-180, and in Harris, J R: “Staging of breast carcinoma” in Harris, J. R., Hellman, S., Henderson, I. C., Kinne D. W. (eds.): Breast Diseases. Philadelphia, Lippincott, 1991. The assigned stage is used as a basis for selection of appropriate therapy and for prognostic purposes. In addition to the TNM parameters, morphologic appearance is used to further classify tumors and thereby aid in selection of appropriate therapy. However, this approach has serious limitations. Tumors with similar histopathologic appearance can exhibit significant variability in terms of clinical course and response to therapy. For example, some tumors are rapidly progressive while others are not. Some tumors respond readily to hormonal therapy or chemotherapy while others are resistant.

Breast carcinoma is the most common cancer in women (31%) and carries the second highest mortality rate exceeded only by that of lung cancer.

For most breast cancers the nodal status is the single most important prognostic indicator and in many cases determines the need for adjuvant chemotherapy. Typically, axillary lymph node dissection (ALND) is performed in order to glean information regarding both staging of the cancer and prognosis. However, since ALND is a surgical procedure, most patients suffer from short term morbidity and some from long term side effects including chronic swelling, discomfort, reduced mobility, and increased risk of infection.

Lymph node metastatic involvement has been subdivided into micro- (0.2-2 mm) and macro-metastases, according to the size of tumor deposits. Numerous studies have demonstrated that the standard pathologic technique of obtaining few hematoxylin & eosin (H&E) stained sections of a lymph node is inadequate for detecting small metastatic deposits. Initial studies did not reveal survival disadvantage in patients with micrometastases and therefore it was generally believed that diligent search for them was of no clinical significance. More recent studies however, have shown this to be no longer tenable. For example, it has been shown that patients with a tumor of 2 cm or less in diameter have poorer disease free survival rates [Dowlatshahi K, et al., Cancer 80:1188-1197]. Accordingly, there is a need for a more detailed and accurate pathologic scrutiny of the lymph nodes.

A new approach for the assessment of breast cancer patients was adopted partially from the malignant melanoma set-up as conceived by Morton in the mid 80's. This approach consisted of lymphatic mapping by injecting a blue dye and an isotope to the tumor area, to be localized in the regional Sentinel Lymph Nodes (SLNs) and then a selective SLN biopsy—for accurately determining ALN status without formal ALND. Thus, complete ALND would be limited to the patients most likely to benefit—those with metastatic nodes. This relies on the concept that tumors spread in an orderly fashion, to the first lymph node (nodes) draining the tumor and subsequently to the ALNs. If the SLNs could be reliably identified and found negative for tumor, the remaining nodes might be predicted negative with a high degree of certainty, thereby avoiding ALND.

There is an approximate 10% chance of obtaining a false negative SLN, (i.e. an SLN negative for tumor with positive nodes found on completion of ALND), with small tumors. It appears that additional definitive factors are needed for the pathological evaluation of the SLN in order to avoid this false negativity.

Turner et al [Turner et al., 1997, Ann of Surg 226:271-276] histopathologically validated the fact that if the SLN is tumor free by serial sectioning and immunohistochemistry using an antibody against a tumor marker (cytokeratin), the probability of non SLN involvement is less than 0.1%. However, the methods described therein are neither practical nor cost effective as a routine clinical procedure.

In a similar fashion, Leers et al, describe the use of flow cytometry as a tool for the detection of micrometastatic tumor cells in the sentinel lymph node procedure of patients with breast cancer using an antibody against cytokeratin to label any epithelial metastatic cells [Leers et al, Journal of Clinical Pathology 2002; 55:359-366].

Altered lymphocyte populations in tumor invaded nodes of breast cancer patients have been identified, suggesting that the presence of metastatic tumor cells in a lymph node is associated with specific alterations in the T cell population [Alam S M, et al., (1993) Immunol Lett 35(3):229-234]. In this study a difference in distribution of T cell subsets, namely CD4/CD8 ratio was identified. It was never suggested by Alam et al, that an alteration in the T cell population of a lymph node may be used as a diagnostic tool for ascertaining lymph node metastasis.

In order to identify specific immune reactivity to breast cancer, both tumor infiltrating lymphocytes and lymphocytes derived from draining lymph nodes were studied for proliferation, phenotype, cytotoxicity, and the ability to secrete cytokines in response to autologous tumor or to polyclonal antigens [Schwartzentruber D J, et al., (1992) J. Immunotherapy 12(1):1-12]. In some cases, a tumor-specific but functionally deficient T-cell immune response in the draining lymph nodes was found. However, Schwartzentruber et al did not ascertain whether the tumor specific responses correlated with metastasized or non-metastasized sub-group of lymph nodes. Accordingly, identifying changes in lymph node T cells was never recommended as a tool for determining lymph node metastasis. Furthermore, the above studies were aimed at identifying an immune response, induced by the lymphocytes, and therefore measured late activation events or cytotoxic activities. In addition, the functional assays were measured in cell suspension, yielding only average signals. Therefore, these assays failed to detect high expression of cells since this was masked by the majority negative population. Hence, the ability to monitor early activation events in sub groups or individual cells is crucial in detecting specific cellular response to tumor cells.

There is thus a widely recognized need for, and it would be highly advantageous to have, a method of ascertaining whether a lymph node is metastatic devoid of the above limitations.

### SUMMARY OF THE INVENTION

According to one aspect of the present invention there is provided a method of determining lymph node metastasis in a subject, the method comprising determining at least one structural or functional parameter of a lymph node cell of the subject, wherein an alteration in the at least one structural or functional parameter with respect to a healthy lymph node cell is indicative of lymph node metastasis.

According to another aspect of the present invention there is provided a method of determining a cancer treatment regimen of a subject comprising; (a) determining at least one structural or functional parameter of a lymph node cell of the subject; and (b) selecting a treatment according to the results of (a), thereby determining a cancer treatment regimen.

According to yet another aspect of the present invention there is provided a kit for determining lymph node metastasis in a subject, the kit comprising a packaging material which comprises at least one agent for determining a structural or functional parameter of a lymph node cell of the subject.

According to still another aspect of the present invention there is provided an article of manufacture comprising packaging material and an agent identified for determining lymph node metastasis in a subject being contained within the packaging material, the agent being capable of determining a structural or functional parameter of a lymph node cell of the subject.

According to an additional aspect of the present invention there is provided a method of diagnosing cancer in a subject, the method comprising determining at least one structural or functional parameter of a lymph node cell of the subject, wherein an alteration in the at least one structural or functional parameter with respect to a healthy lymph node cell is indicative of cancer, thereby diagnosing cancer in a subject.

According to further features in preferred embodiments of the invention described below, the lymph node cell is a lymphocyte.

According to still further features in the described preferred embodiments, the lymph node cell is viable.

According to still further features in the described preferred embodiments, the lymph node cell is non-malignant.

According to still further features in the described preferred embodiments, the lymph node cell is isolated.

According to still further features in the described preferred embodiments, the structural parameter of said lymph node cell is a cell size, an organelle size, a cell number and an organelle number.

According to still further features in the described preferred embodiments, the cell size above a predetermined threshold is indicative of lymph node metastasis.

According to still further features in the described preferred embodiments, the functional parameter of the cell is at least one of the following:

i. an expression level or activity of an enzyme;

ii an expression level or activity of a cell surface marker;

iii. an expression level or activity of a chemokine receptor;

iv. an expression level or activity of a of a chemokines; and/or

v. a proliferation rate.

According to still further features in the described preferred embodiments, the enzymatic activity is an esterase activity or a metalloproteinase activity.

According to still further features in the described preferred embodiments, the metalloproteinase activity is an MMP2 activity or an MMP9 activity.

According to still further features in the described preferred embodiments, a homogeneous expression of the cell surface marker is indicative of lymph node metastasis.

According to still further features in the described preferred embodiments, the cell surface marker is selected from the group consisting of CD71, CD25, CD4, CD8, CD69, CD20, CD19, CD3 HLA-DR and CD138.

According to still further features in the described preferred embodiments, the method further comprises contacting the lymph node cell of the subject and/or the healthy lymph node cell with a tumor cell prior to determining the at least one structural or functional parameter.

According to still further features in the described preferred embodiments, the contacting is effected for about 18 hours.

According to still further features in the described preferred embodiments, the tumor tissue is autologous tumor tissue.

According to still further features in the described preferred embodiments, the tumor cell is viable.

According to still further features in the described preferred embodiments, the lymph node is a breast lymph node.

According to still further features in the described preferred embodiments, the lymph node is a sentinel lymph node.

According to still further features in the described preferred embodiments, the breast lymph node is an axillary lymph node.

According to still further features in the described preferred embodiments, the at least one agent for determining a functional parameter of the lymph node cell is a substrate for an enzyme.

According to still further features in the described preferred embodiments, the enzyme is an esterase or a metalloproteinase.

According to still further features in the described preferred embodiments, the at least one agent is fluoresceindiacetate or DQ Gelatin FITC.

According to still further features in the described preferred embodiments, the at least one agent for determining a functional parameter of said cell is an antibody against a cell surface marker.

According to still further features in the described preferred embodiments, the method further comprises contacting cells of interest with said lymph node cell prior to said determining.

The present invention successfully addresses the shortcomings of the presently known configurations by providing a novel method for determining lymph node metastasis in a subject without the need to identify tumor cells in that lymph node.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. All publications, patent applications, patents, and other references mentioned herein are incorporated by reference in their entirety. In case of conflict, the patent specification, including definitions, will control. In addition, the materials, methods, and examples are illustrative only and not intended to be limiting.

### BRIEF DESCRIPTION OF THE DRAWINGS

The invention is herein described, by way of example only, with reference to the accompanying drawings. With specific reference now to the drawings in detail, it is stressed that the particulars shown are by way of example and for purposes of illustrative discussion of the preferred embodiments of the present invention only, and are presented in the cause of providing what is believed to be the most useful and readily understood description of the principles and conceptual aspects of the invention. In this regard, no attempt is made to show structural details of the invention in more detail than is necessary for a fundamental understanding of the invention, the description taken with the drawings making apparent to those skilled in the art how the several forms of the invention may be embodied in practice.

In the drawings:

FIGS. 1A-B are distribution histograms of cell area measured in individual cell populations derived from tumor-free or metastatic lymph nodes. FIG. 1A illustrates the greater cell area of individual cell populations in metastatic lymph nodes compared to tumor-free lymph nodes. FIG. 1B illustrates the increase in cell area of individual cell populations in metastatic lymph nodes compared to tumor-free lymph nodes following autologous tumor tissue (TT) activation.

FIGS. 2A-B are distribution histograms of intracellular reaction rates measured in individual cell populations derived from tumor free or metastatic lymph nodes prior to (FIG. 2A) and following (FIG. 2B) autologous tumor cell activation.

FIG. 3 is a bar graph illustrating that the percent of individual metastatic lymph node cells with FDA slopes greater than 1.5 doubled following autologous tumor tissue activation.

FIGS. 4A-B are scatter plots illustrating that the intracellular reaction rates of small cells increase to a greater extent than the intracellular reaction rates of large cells in both tumor free and metastatic lymph nodes following autologous tumor cell activation.

FIGS. 5A-B are scatter plots correlating the heterogeneity of CD71 with cell area in both tumor free and metastatic lymph nodes. FIG. 5A illustrates that cells derived from tumor-free lymph node displayed a heterogenous CD71 expression which became even more heterogeneous following activation. As illustrated in FIG. 5B, cells derived from metastatic lymph nodes exhibited a relatively homogeneous CD71 expression on their cell surface. An increase in heterogeneity was observed following activation with autologous tumor tissue.

FIG. 6 is a bar graph illustrating the correlation between the cell's ability to hydrolyze FDA and expression of early lymphocyte activation marker (CD71) on its cell surface. Cells with higher CD71 expression exhibited lower FDA reaction rate. This effect was more pronounced following autologous tumor tissue activation. The FDA reaction rate increased significantly following autologous tumor tissue activation for cells with both high and low CD71 expression.

FIGS. 7A-B are bar graphs illustrating MMP activity (Mean Intensity measured 2 h and 24 h culture within LiveCell array) in individual cells originating from tumor free or metastatic LNs with or without TT activation. Cells derived from metastatic lymph nodes exhibited higher MMP activity both prior to and following activation with autologous tumor tissue.

### DESCRIPTION OF THE PREFERRED EMBODIMENTS

The present invention is of a method of determining lymph node metastasis in a subject. Specifically, the present invention can be used to select the most favorable regimen for the treatment of cancer.

The principles and operation of the present invention may be better understood with reference to the drawings and accompanying descriptions.

Before explaining at least one embodiment of the invention in detail, it is to be understood that the invention is not limited in its application to the details set forth in the following description or exemplified by the Examples. The invention is capable of other embodiments or of being practiced or carried out in various ways. Also, it is to be understood that the phraseology and terminology employed herein is for the purpose of description and should not be regarded as limiting.

A major challenge in cancer treatment lies in the attempt to provide accurate diagnostic, prognostic, and predictive information. For most cancers the nodal status is the single most important prognostic indicator and in many cases determines the need for adjuvant chemotherapy. Typically, lymph node metastatic involvement is determined by identifying metastasized tumor cells in hematoxylin & eosin (H&E) or cytokeratin stained sections of a lymph node. Numerous studies however, have demonstrated that these standard pathologic techniques are inadequate for detecting small metastatic deposits. In addition there is a 10% chance of obtaining a false negative SLND, (i.e. an SLN negative for tumor with positive nodes found on completion of ALND), with small tumors.

While reducing the present invention to practice, the present inventors have uncovered a novel approach for determining lymph node metastasis in a subject. Accordingly, the present inventors have shown that it is possible to use alterations in the native lymph node cells as a diagnostic tool for ascertaining lymph node metastasis without the need to identify tumor cells in that lymph node.

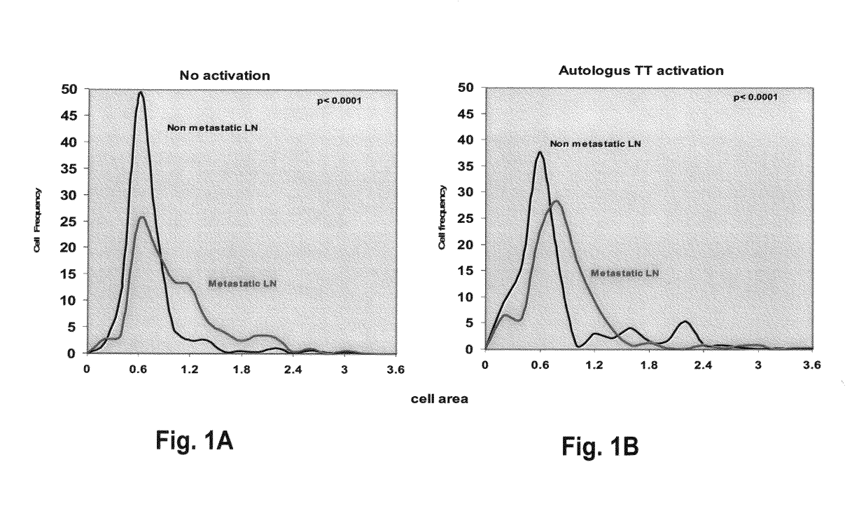

As illustrated in the Examples section hereinbelow, both structural and functional parameters of lymph node cells may be used as markers for metastasis. Thus, the present inventors have shown that there is a significant difference between the cell size of tumor-free and metastatic lymph nodes, the latter exhibiting a larger image area. In addition, metastatic lymph nodes exhibited a greater increase in cell area following autologous tumor tissue (TT) activation than tumor-free lymph nodes (FIGS. 1A-B).

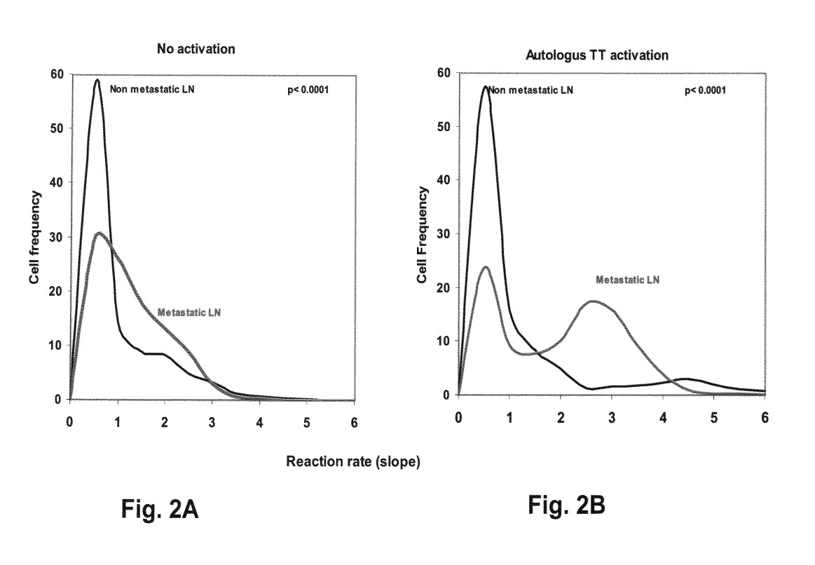

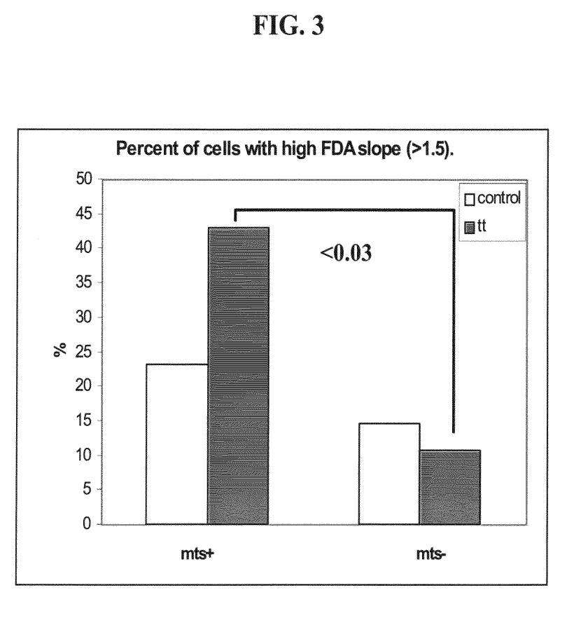

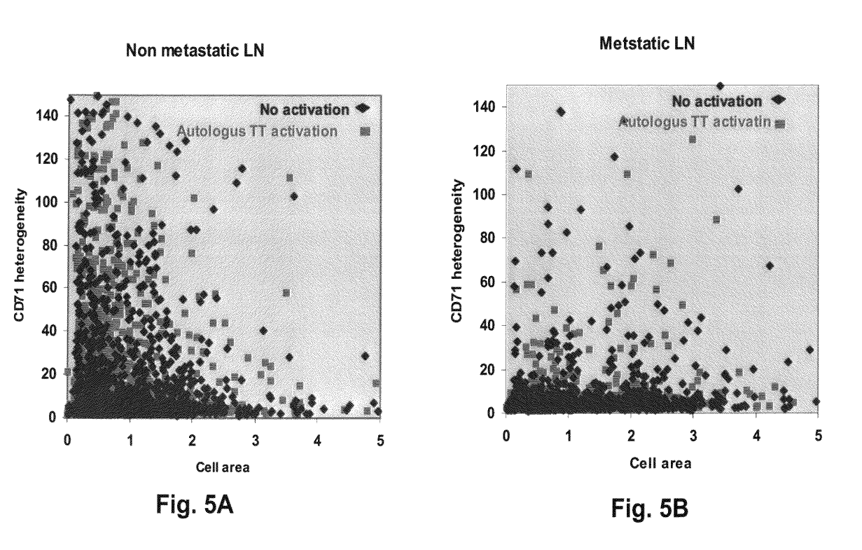

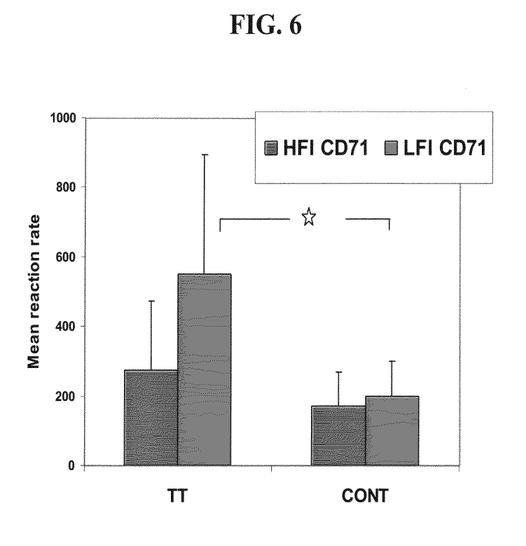

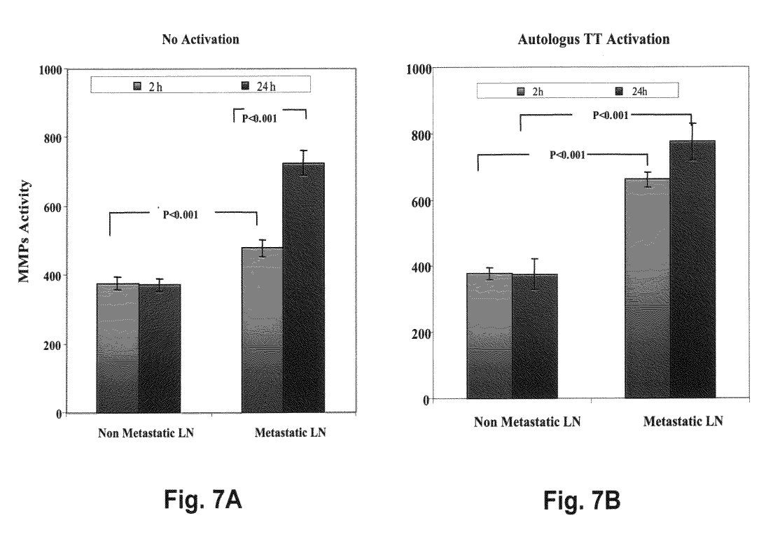

The rate of hydrolysis of intracellular esterase was shown to be higher in cells of a metastatic lymph node than a tumor-free lymph node, a phenomenon that was exacerbated following autologous tumor tissue activation (FIGS. 2A-B and FIG. 3). In addition, the present inventors have shown that cells derived from metastatic lymph nodes exhibited a different pattern of CD71 expression on their cell surface than cells derived from tumor-free lymph nodes both prior to and following tumor tissue activation (FIGS. 5A-B and FIG. 6). Furthermore, it was shown that cells derived from metastatic lymph nodes exhibited higher MMP activity than cells derived from tumor-free lymph nodes both prior to and following activation with autologous tumor tissue (FIGS. 7A-B).

Altered lymphocyte populations in tumor invaded nodes of breast cancer patients have been identified previously [Alam S M, et al., (1993) Immunol Lett 35(3):229-234]. In this study a difference in distribution of T cell subsets, namely CD4/CD8 ratio was identified. In sharp contrast to the present invention, Alam et al, never suggested using this difference to serve as a diagnostic tool for ascertaining lymph node metastasis.

Furthermore, Alam et al, unlike the present inventors do not mention monitoring cellular activity prior to or following incubation in the presence of tumor antigens. This type of analysis ensures the specificity of the results since changes in T-cell subsets may occur due to immune deregulations and are not specific to tumor invasion.

Schwartzentruber et al [Schwartzentruber D J, et al., (1992) J. Immunotherapy 12(1):1-12] identified a tumor-specific but functionally deficient T-cell immune response in the draining lymph nodes of some breast cancer patients. Schwartzentruber and co-workers did not ascertain whether this tumor specific response correlated with a metastasized or non-metastasized sub-group of lymph nodes. Accordingly, identifying changes in lymph node T cells was never recommended as a tool for determining lymph node metastasis.

Thus, according to one aspect of the present invention there is provided a method of determining lymph node metastasis in a subject, the method comprising determining at least one structural or functional parameter of a lymph node cell of the subject, wherein an alteration in the at least one structural or functional parameter with respect to a healthy lymph node cell is indicative of lymph node metastasis.

As used herein, the phrase “lymph node” refers to a mass of lymphatic tissue that is surrounded by a capsule of connective tissue. Typically lymph nodes are found in the neck, around the collarbone, in the armpit and in the groin. The lymph from the lower limbs drains through deep and superficial nodes including popliteal nodes and inguinal nodes.

Selection of the lymph node is dependent on the site of the primary tumor. Thus, a preferred lymph node tissue for detecting cancer metastasis would be the sentinel lymph node. As used herein the phrase “sentinel lymph node” refers to the nearest lymph node to the site of the neoplastic tissue and within the pertinent lymph drainage basin. Such a node, being on the most direct drainage pathway, will present the most likely site of early metastasis. Methods of locating a sentinel lymph node are described hereinbelow.

According to another embodiment, the lymph node is a breast lymph node i.e. one that drains the breast tissue. An exemplary breast lymph node is an axillary lymph node. As used herein the phrase “axillary lymph node” refers to a lymph node in the armpit region that drains lymph channels from the breast.

Lymph nodes may be located using any method known in the art. One such method of detection is described in U.S. Pat. No. 4,827,945 to Groman et al which describes the use of superparamagnetic metal oxide materials exhibiting certain magnetic and biological properties which make them uniquely suitable for use as magnetic resonance imaging (MRI) agents to enhance MRI images of human organs and tissues. These agents may be coated with biological molecules to target specific organs or tissues such that, upon administration to an animal, the agents are collected in the target organs. The MRI agents are administered by various routes but are typically injected directly into the animal's bloodstream.

In addition, radiation-based methods for locating lymph nodes are known. For example, U.S. Pat. No. 5,732,704 to Thurston et al. describes a method for identifying a sentinel node located within a grouping of regional nodes at a lymph drainage basin associated with neoplastic tissue such as those found in cancerous tumors. The method, like other radiation-based detection methods, requires a radiopharmaceutical to be injected at the site of the neoplastic tissue. This radiopharmaceutical migrates along a lymph duct toward the drainage basin containing the sentinel node. The sentinel node is located where detected radiation intensity is at a local maximum.

As used herein, the phrase “lymph node cell” refers to a lymph node cell present in the lymph node that has not been released into the blood (i.e. not a circulating lymph node cell). Preferably, the lymph node is encapsulated.

Examples of lymph node cells include, but are not limited to lymphocytes, dendritic cells (DC), macrophages and blast cells. According to a preferred embodiment of this aspect of the present invention the lymph node cells are non-malignant.

As used herein, the phrase “lymph node metastasis” refers to a process by which tumor cells from the site of a primary tumor enters the lymph node. The tumor cells may be present or absent in the lymph node during the time of determining whether a metastatic event has occurred.

As used herein a “subject” refers to a mammal, preferably a human subject. Examples of non-human mammals include, but are not limited to, mouse, rat, rabbit, bovine, porcine, ovine, equine, canine and feline. Typically, the subject has been diagnosed with cancer or is undergoing diagnostic evaluation.

Determining alterations between the lymph node cell of a test subject and a control lymph node cell can be effected ex-vivo (e.g. in vitro) or in vivo according to this aspect of the present invention. According to a preferred embodiment, the method of the present invention is effected ex-vivo.

Lymph nodes are typically retrieved by a surgical biopsy. The surgical biopsy procedure may include any biopsy procedure presently known in the art. Examples of such procedures which may be used to remove lymph nodes include, but are not limited to a fine-needle aspiration biopsy, a core needle biopsy and an open biopsy. When the removed lymph node is not a sentinel lymph node preferably more than one lymph node is removed. U.S. Pat. No. 5,961,458 to Carroll describes a minimally invasive surgical probe for detecting and removing radioactively tagged tissue, e.g., a sentinel lymph node within the body of a living being.

Any method may be used for obtaining cells from a lymph node provided that following which the cells are in a state such that they may be analyzed according to the method of the present invention.

Lymph node cells may be obtained from a lymph node in situ during a surgical procedure (e.g. by shearing) or alternatively, the lymph node or part thereof may be removed as described hereinabove and the lymph node cells obtained afterwards.

According to a preferred embodiment of this aspect of the present invention the lymph node cells are isolated. As used herein, the term “isolated” refers to lymph node cells that have been removed from their naturally-occurring in-vivo location (e.g. armpit or groin). Preferably the isolated lymph node cells are substantially free from other substances (e.g., extracellular matrix, other cells.) that are present in its in-vivo location. The isolated lymph node cells may be individually isolated and dispersed into a single cell suspension—e.g. by the addition of trypsin or by trituration.

According to a preferred embodiment of this aspect of the present invention, the lymph node cells are viable. An exemplary method for obtaining viable lymph node cells from a lymph node is described in Example 1 hereinbelow. Essentially, the cells from the internal side of a lymph node are sheared using a syringe filled with medium (e.g. RPMA-1640). During this process, a constant pressure applied to the syringe piston produces a steady stream of fluid that hits the tissue and extracts cells from the lymph node.

Cells from the lymph node tissue may be analyzed immediately or alternatively the lymph node tissue may be frozen and cells analyzed at a later stage when analysis is required. Methods of freezing tissues and cells are well known in the art. Subsequently, the lymph node tissue may be processed according to the particular method of detection of metastasis. For example, lymph node cells may be processed so as to remain intact or its membrane disintegrated (such as following lysis or sonication) so as to release the inner cell components. Alternatively, lymph node cells may be cultured. Standard culturing techniques are also well known in the art. Cells may be fixed on slides using standard procedures for ex vivo analysis. Fixing acts to preserve cells in a reproducible and life-like manner, following their removal in a surgical biopsy procedure. Fixation methods are well known in the art. These methods typically rely on either crosslinking agents, such as paraformaldehyde, or rapidly dehydrating agents, such as methanol.

As mentioned above, the method of the present invention is based on the finding that lymph node cells display an altered phenotype following interaction with a cancerous cell as compared to a healthy lymph node cell.

A healthy lymph node cell may be taken from a healthy subject or from a non-healthy subject (i.e. patient) with an unaffected lymph node or from the subject itself (from nodes which are known not to encounter a cancer cell). Since the above-mentioned structural or functional parameters depend on, amongst other things, species, age and cell type, it is preferable that the unaffected control cells come from a subject of the same species, age and from the same sub-lymph node tissue. Alternatively, control data may be taken from databases and literature. It is preferable that at least one cell is analyzed in accordance with the present invention. However the present invention certainly envisages the analysis of few cells to whole tissues.

According to this aspect of the present invention, the altered phenotype may comprise a structural or functional parameter.

The phrase “a structural parameter” as used herein, refers to the presence or absence of a quality or quantity of a cell or subcellular structure (organelle, or biomolecule, e.g., protein, nucleic acid, carbohydrate, lipid) or morphology which may be used to distinguish between a lymph node cell that has encountered a cancerous cell and a lymph node cell that has not. Examples of structural parameters according to this aspect of the present invention include but are not limited to a cell size, cell morphology, cytoskeleton organization, cell motility an organelle size, a cell number and an organelle number.

The phrase “a functional parameter” as used herein, refers to a quantitative or qualitative cell characteristic which correlates with its operation that may be used to distinguish between a lymph node cell that has encountered a cancerous cell and a lymph node cell that has not.

Preferably, the functional parameter is a functional kinetic parameter (e.g. reaction rate, or MMPs activity as described herein below) since kinetic parameters are more sensitive and reflect early activation events of the cells.

It will be appreciated that functional parameters may overlap with structural parameter e.g., presence of secretory vesicles. Examples of functional parameters according to this aspect of the present invention include, but are not limited an enzyme expression and/or activity; a cell surface marker expression and/or activity; a chemokine receptor expression and/or activity; a chemokine expression and/or activity; and/or a proliferation rate.

As used herein, the phrase “an alteration in the at least one structural or functional parameter” refers to measuring a quantitative or qualitative difference in a structural or functional parameter between the lymph node cells of the test subject and a normal control subject.

Agents which may be used to analyze such parameters are described hereinbelow.

Measuring alterations in at least one structural or functional parameter may be preceded by contacting the test subjects and/or control lymph node cells with tumor cells. The present inventors have shown that this step may serve to highlight alterations in such structural or functional parameters. Preferably the tumor cells are autologous (i.e. removed from the same patient from whom the lymph node cells were obtained). Preferably the tumor cells are viable. The tumor cells may be used as isolated cells or as cells comprised in a tumor tissue.

An exemplary method of contacting lymph node cells with tumor cells is described in Example 1 of the Examples section herein below. Accordingly, the lymph node cells were contacted with intact un-fractionated autologous tumor tissue for at least 18 hours at 37° C. in the presence of 5% CO₂ prior to measuring the structural or functional parameters of the present invention.

As mentioned herein above, a functional parameter of a lymph node cell may be used as a gauge for determining whether metastasis has occurred in the lymph node.

As used herein, the term “chemokine” refers to a member of a group of proteins that act as chemoattractants for host defense effector cells such as neutrophils, monocytes and lymphocytes (see, for example, Rollins, B. J. (1997) Blood 90(3):909-928, and Baggiolini, M. (1998) Nature 392:565-568).

As used herein, the term “chemokine receptor” refers to a polypeptide that is able to bind a chemokine and/or mediate intracellular signalling. A non-limiting list of representative chemokine receptors includes CCR1, CCR2, CCR3, CCR4, CCR5, CCR6, CCR7, CCR8, CCR9, CXCR1, CXCR2, CXCR3, CXCR4, CXCR5, CXCR6, CXXXCR1 and XCR1.

According to one embodiment of the present invention, the rate of hydrolysis of intracellular esterase may be used as a marker for a metastatic lymph node. A significantly higher rate of esterase hydrolysis in test lymph node cells compared to control lymph node cells is indicative of a metastasis (FIGS. 2A-B and FIG. 3). Since this effect is exacerbated in the presence of tumor cells (as described hereinabove), it may be preferable to perform this assay following contacting with such cells.

According to another embodiment of the present invention, an MMP activity (e.g. MMP2 or MMP9) may be assayed for activity wherein a significantly higher MMP activity in a test lymph node cell compared to an MMP activity in a healthy lymph node cell is indicative of a metastatic lymph node (FIGS. 7A-B). Since this effect is exacerbated in the presence of tumor cells (as described hereinabove), it may be preferable to perform this assay following contacting with such cells.

An expression or activity of a cell surface marker may also be used as an indicator for a metastatic lymph node.

The phrase “cell surface marker” refers to a distinguishing protein, carbohydrate, or glycoprotein present on the surface of a cell. Examples of cell surface markers include but are not limited to CD71, CD25, CD4, CD8 and CD69.

The present inventors have shown that cells derived from metastatic lymph nodes exhibit a different pattern of CD71 expression on their cell surface than cells derived from tumor-free lymph nodes both prior to and following tumor tissue activation. Thus according to another embodiment of this aspect of the present invention, the homogeneity of expression of a cell surface marker may be used as an indicator of lymph node metastasis, wherein a significantly more homogeneous expression in a test lymph node cell compared to an expression in a healthy lymph node cell is indicative of a metastatic lymph node (FIGS. 5A-B and FIG. 6).

As mentioned herein above, a structural parameter of a test lymph node cell may be used as a gauge for determining whether metastasis has occurred in the lymph node.

According to a particular embodiment of this aspect of the present invention, metastasis may be considered to have occurred if the size of the lymph node cells is greater [i.e., statistically greater, as can be determined by analysis of variance (e.g., t-Test)] per cell compared with healthy control lymph node cells. According to another embodiment, metastasis may be considered to have occurred if tumor tissue acts to increase the test lymph node cell size to a significantly greater extent than it increases normal lymph node cell size—see FIGS. 1A-B.

Agents which may be used to determine changes in the functional and structural parameters described herein above include antibodies (e.g. to determine expression levels and patterns of expression of proteins of interest); lectins, chemicals (e.g. to determine activity levels of proteins of interest); dyes (e.g. to determine the structural parameters of the present invention; and polynucleotides (e.g. to determine expression levels of proteins of interest).

The term “antibody” as used in this invention includes intact molecules as well as functional fragments thereof, such as Fab, F(ab′)2, and Fv that are capable of binding to the specific lymph node cell proteins. Smaller antibody fragments may be advantageous over whole antibodies since they are able to penetrate tissue more readily and are more rapidly cleared from the body. This is especially relevant for the in-vivo use of antibodies.

Antibodies may be generated via any one of several methods known in the art, which methods can employ induction of in-vivo production of antibody molecules, screening of immunoglobulin libraries [Orlandi D. R. et al., 1989. Proc. Natl. Acad. Sci. U.S.A. 86:3833-3837; Winter G. et al., 1991. Nature 349:293-299] or generation of monoclonal antibody molecules by continuous cell lines in culture. These include, but are not limited to, the hybridoma technique, the human B-cell hybridoma technique, and the Epstein-Barr virus (EBV)-hybridoma technique [Kohler G. et al., 1975. Nature 256:495-497; Kozbor D. et al., 1985. J. Immunol. Methods 81:31-42; Cote R J. et al., 1983. Proc. Natl. Acad. Sci. U.S.A. 80:2026-2030; Cole S P. et al., 1984. Mol. Cell. Biol. 62:109-120].

Antibody fragments can be obtained using methods well known in the art. [see, for example, Harlow and Lane, “Antibodies: A Laboratory Manual”, Cold Spring Harbor Laboratory, New York, (1988)].

It will be appreciated that for in vivo use, humanized antibodies are preferably used. Methods for humanizing non human antibodies are well known in the art. See, for example: Jones et al., 1986. Nature 321:522-525; Riechmann et al., 1988. Nature 332:323-327; Verhoeyen et al., 1988. Science 239:1534-1536; U.S. Pat. No. 4,816,567

Once antibodies are obtained, they may be tested for activity, for example via ELISA.

The antibodies may be labeled with a detectable moiety which can be quantified either directly or indirectly. Examples of detectable moieties that can be used in the present invention include but are not limited to radioactive isotopes, phosphorescent chemicals, chemiluminescent chemicals, fluorescent chemicals, enzymes, fluorescent polypeptides and epitope tags. Alternatively, the antibodies may be detected using a second antibody which itself is conjugated to a detectable moiety.

The detectable moiety may be attached to the antibody using any suitable chemical linkage, direct or indirect, as via a peptide bond (when the detectable moiety is a polypeptide), or via covalent bonding to an intervening linker element, such as a linker peptide or other chemical moiety, such as an organic polymer. Such chimeric antibodies may be linked via bonding at the carboxy (C) or amino (N) termini of the antibodies, or via bonding to internal chemical groups such as straight, branched or cyclic side chains, internal carbon or nitrogen atoms, and the like. Such modified antibodies can be easily identified and prepared by one of ordinary skill in the art, using well known methods of peptide synthesis and/or covalent linkage of peptides. Description of fluorescent labeling of antibodies is provided in details in U.S. Pat. Nos. 3,940,475, 4,289,747, and 4,376,110.

When the detectable moiety is a polypeptide, the chimeric antibodies thereof may be produced by recombinant means or may be chemically synthesized by, for example, the stepwise addition of one or more amino acid residues in defined order using solid phase peptide synthetic techniques. Examples of polypeptide moieties that can be linked to antibodies using recombinant DNA technology include fluorescent polypeptides, phosphorescent polypeptides, enzymes and epitope tags. Expression vectors can be designed to fuse antibodies encoded by the heterologous nucleic acid insert to fluorescent polypeptides. For example, antibodies can be expressed from an expression vector fused with a green fluorescent protein (GFP)-like polypeptide. A wide variety of vectors are commercially available that fuse proteins encoded by heterologous nucleic acids to the green fluorescent protein from Aequorea victoria (“GFP”), the yellow fluorescent protein and the red fluorescent protein and their variants (e.g., Evrogen). In these systems, the fluorescent polypeptide is entirely encoded by its amino acid sequence and can fluoresce without requirement for cofactor or substrate. Expression vectors that can be employed to fuse proteins encoded by the heterologous nucleic acid insert to epitope tags are commercially available (e.g., BD Biosciences, Clontech).

Detectable antibodies to cell surface markers are commercially available e.g. anti-human CD71R-phycoerythrin (PE) may be purchased from Chemicon Europe, U.K and anti-human CD69 PE-conjugated antibodies may be purchased from Bioscience.

As mentioned hereinabove, chemicals may be used to determine activity levels of proteins of interest. For example, as illustrated in the Examples section below an esterase activity may be measured in lymph node cells utilizing fluoresceindiacetate (FDA) as a substrate and a metalloproteinase activity may be measured in lymph node cells utilizing a DQ Gelatin FITC conjugated substrate (EnzChek; Molecular probes, IS).

As mentioned, dyes may also be used to determine structural parameters of the present invention. Examples of dyes which may be used to estimate cell size include, but are not limited to the following: Alexa fluor 488 phalloidin (Molecular Probes, Oregon), Chloromethylfluorescein diacetate (CMFDA), calcein AM, BCECF AM, Rhodamine dextran, Dihexadecyl tetramethylindocarbocyanine perchlorate (DiIC16) lipid dyes and Triethylammonium propyl dibutylamino styryl pyridinium (FM 4-64, FM 1-43) lipid dyes.

Live cells stained with DiIC16 have homogeneously labeled plasma membranes, and the projected cross-sectional area of the cell is uniformly discriminated from background by fluorescence intensity of the dye. Live cells stained with cytosolic dyes such as CMFDA produce a fluorescence intensity that is proportional to cell thickness. Although cell labeling is dimmer in thin regions of the cell, total cell area can be discriminated from background. Fixed cells can be stained with cytoskeletal dyes such as Alexa fluor 488 phalloidin that label polymerized actin. Phalloidin does not homogeneously stain the cytoplasm, but still permits discrimination of the total cell area from background.

Cell number and cell proliferation may also be measured using a dye. For cell number, the total number of cells may be counted or alternatively the number of apoptotic or dead cells may be counted. For cell proliferation, the total number of proliferating cells may be counted. Exemplary dyes which may be used for these purposes are described herein below. As with the dyes used for measuring cellular size, probes for measuring cell number are commercially available from a number of sources e.g. Molecular Probes (Eugene, Oreg.) and/or Sigma Chemical Co. (St. Louis, Mo.).

Trypan blue is able to differentiate live cells from dead or dying cells since it easily diffuses across cell membrane of dead or dying cells, but cannot cross membranes of live cells (Evans). Cell counting is typically performed visually by microscopy or using automated counters. Incorporation of H3-thymidine into DNA during cell growth is another method of counting live cells.

Tetrazolium salts, including MTT and XTT formazan dyes and Brdu may be used to assay cell proliferation, cell viability, and/or cytotoxicity. The tetrazolium salts are converted in metabolically active cells by cytoplasmic enzymes, generating a staining. The reaction is attributed mainly to mitochondrial enzymes and electron carriers, but a number of other non-mitochondrial enzymes have been implicated.

Organelle size and number may also be measured using an organelle-specific dye. A wide range of organelle specific dyes are known in the art. [For example see “Handbook of Fluorescent Probes and Research Chemicals” www.probes.com/handbook/sections/1202.html), Chapter 12—Probes for Organelles].

As mentioned, polynucleotides may also be used to measure the expression level of proteins of interest by base pairing to a nucleic acid sequence (i.e., DNA or RNA) encoding the protein of interest.

There are three types of oligonucleotide probes that may be used for the detection of nucleic acids—single stranded DNA probes, double stranded DNA probes and RNA probes.

The term “oligonucleotide” refers to a single stranded or double stranded oligomer or polymer of ribonucleic acid (RNA) or deoxyribonucleic acid (DNA) or mimetics thereof. This term includes oligonucleotides composed of naturally-occurring bases, sugars and covalent internucleoside linkages (e.g., backbone) as well as oligonucleotides having non-naturally-occurring portions which function similarly to respective naturally-occurring portions.

Oligonucleotides designed according to the teachings of the present invention can be generated according to any oligonucleotide synthesis method known in the art such as enzymatic synthesis or solid phase synthesis. Equipment and reagents for executing solid-phase synthesis are commercially available from, for example, Applied Biosystems. Any other means for such synthesis may also be employed; the actual synthesis of the oligonucleotides is well within the capabilities of one skilled in the art and can be accomplished via established methodologies as detailed in, for example, “Molecular Cloning: A laboratory Manual” Sambrook et al., (1989); “Current Protocols in Molecular Biology” Volumes I-III Ausubel, R. M., ed. (1994); Ausubel et al., “Current Protocols in Molecular Biology”, John Wiley and Sons, Baltimore, Md. (1989); Perbal, “A Practical Guide to Molecular Cloning”, John Wiley & Sons, New York (1988) and “Oligonucleotide Synthesis” Gait, M. J., ed. (1984) utilizing solid phase chemistry, e.g. cyanoethyl phosphoramidite followed by deprotection, desalting and purification by for example, an automated trityl-on method or HPLC.

The oligonucleotide of the present invention are typically at least 17, at least 18, at least 19, at least 20, at least 22, at least 25, at least 30 or at least 40, bases specifically hybridizable with nucleic acid sequences encoding proteins of interest described hereinabove.

It will be appreciated that in vivo use of oligonucleotides may necessitate the inclusion of base and/or backbone modifications as described herein below, which may increase bioavailability and reduce cytotoxicity.

For example, the oligonucleotides of the present invention may comprise heterocylic nucleosides consisting of purines and the pyrimidines bases, bonded in a 3′ to 5′ phosphodiester linkage.

It is possible to radiolabel all types of oligonucleotides with ¹⁸-Fluorine, ³⁵S, ³H, ¹⁴C and ⁹⁹Tc. Isotopic phosphorus is unfavorable because its very short half life makes analysis difficult. Another disadvantage with oligonucleotides labeled on the 3′ or 5′ terminus with ³²P is that the label will be rapidly hydrolyzed by exonucleases that are abundant in the blood. ³⁵S has a higher energy, and therefore is easier to detect at low levels. Tritium is a relatively weak isotope, limiting levels of detection and analytical options. Radiolabeled sulfur obviously lends itself perfectly to use with phosphorothioate (PS) oligonucleotides. Although a single radioactive phosphorothioate link can be added to any non-PS oligonucleotide, an overt change to the molecular composition is made which defeats one advantage of using isotopes. Tritium, on the other hand, can be placed in any oligonucleotide, including phosphorothioates, with no change to molecular structure. Other radioisotopes that can be used to label oligonucleotides include, but are not limited to ¹⁸-Fluorine, ¹⁴C and ⁹⁹Tc.

Oligonucleotides can also be fluorescence labeled. Fluorescent labeled dUTPS are commercially available (e.g., fluorescein 12-dideoxyuridine-5′-triphosphate oligodeoxyribonucleotides—Boehringer Mannheim (Germany) and may be incorporated in the oligonucleotide using the enzyme terminal deoxynucleotidyl transferase (Life Technologies, Inc.) Fluorescent labeled oligonucleotides such as Rhodamine X labeled oligonucleotides may be ordered commercially from Companies such as Takara Shuzo (Kyoto, Japan) and Metabion, Planegg-Martinsried. Phosphorescent dUTPs may also be prepared [De Haas, R., 1999, Journal of Histochemistry and Cytochemistry, Vol. 47, 183-196] and used to produce phosphorescent labeled oligonucleotides.

The prior art teaches of a number of delivery strategies which can be used to efficiently deliver oligonucleotides into a wide variety of cell types [see, for example, Luft J Mol Med 76: 75-6 (1998); Kronenwett et al., Blood 91: 852-62 (1998); Rajur et al., Bioconjug Chem 8: 935-40 (1997); Lavigne et al., Biochem Biophys Res Commun 237: 566-71 (1997) and Aoki et al., (1997) Biochem Biophys Res Commun 231: 540-5 (1997)].

The following is a non-exhaustive list of methods which may be used for the ex-vivo analysis of the structural and functional parameters of the present invention using the agents described hereinabove. It will be appreciated the described methods that enable the analysis of individual cells may be preferable over methods which analyze populations of cells since, in the latter, individual events may be masked by a majority negative population.

The expression level of the RNA in the cells of the present invention can be determined using methods known in the arts.

Northern Blot analysis: This method involves the detection of a particular RNA in a mixture of RNAs. An RNA sample is denatured by treatment with an agent (e.g., formaldehyde) that prevents hydrogen bonding between base pairs, ensuring that all the RNA molecules have an unfolded, linear conformation. The individual RNA molecules are then separated according to size by gel electrophoresis and transferred to a nitrocellulose or a nylon-based membrane to which the denatured RNAs adhere. The membrane is then exposed to labeled DNA probes. Probes may be labeled using radio-isotopes or enzyme linked nucleotides. Detection may be using autoradiography, colorimetric reaction or chemiluminescence. This method allows both quantitation of an amount of particular RNA molecules and determination of its identity by a relative position on the membrane which is indicative of a migration distance in the gel during electrophoresis.

RT-PCR analysis: This method uses PCR amplification of relatively rare RNAs molecules. First, RNA molecules are purified from the cells and converted into complementary DNA (cDNA) using a reverse transcriptase enzyme (such as an MMLV-RT) and primers such as, oligo dT, random hexamers or gene specific primers. Then by applying gene specific primers and Taq DNA polymerase, a PCR amplification reaction is carried out in a PCR machine. Those of skills in the art are capable of selecting the length and sequence of the gene specific primers and the PCR conditions (i.e., annealing temperatures, number of cycles and the like) which are suitable for detecting specific RNA molecules. It will be appreciated that a semi-quantitative RT-PCR reaction can be employed by adjusting the number of PCR cycles and comparing the amplification product to known controls.

RNA in situ hybridization stain: In this method DNA or RNA probes are attached to the RNA molecules present in the cells. Generally, the cells are first fixed to microscopic slides to preserve the cellular structure and to prevent the RNA molecules from being degraded and then are subjected to hybridization buffer containing the labeled probe. The hybridization buffer includes reagents such as formamide and salts (e.g., sodium chloride and sodium citrate) which enable specific hybridization of the DNA or RNA probes with their target mRNA molecules in situ while avoiding non-specific binding of probe. Those of skills in the art are capable of adjusting the hybridization conditions (i.e., temperature, concentration of salts and formamide and the like) to specific probes and types of cells. Following hybridization, any unbound probe is washed off and the slide is subjected to either a photographic emulsion which reveals signals generated using radio-labeled probes or to a colorimetric reaction which reveals signals generated using enzyme-linked labeled probes.

In situ RT-PCR stain: This method is described in Nuovo G J, et al. [Intracellular localization of polymerase chain reaction (PCR)-amplified hepatitis C cDNA. Am J Surg Pathol. 1993, 17: 683-90] and Komminoth P, et al. [Evaluation of methods for hepatitis C virus detection in archival liver biopsies. Comparison of histology, immunohistochemistry, in situ hybridization, reverse transcriptase polymerase chain reaction (RT-PCR) and in situ RT-PCR. Pathol Res Pract. 1994, 190: 1017-25]. Briefly, the RT-PCR reaction is performed on fixed cells by incorporating labeled nucleotides to the PCR reaction. The reaction is carried on using a specific in situ RT-PCR apparatus such as the laser-capture microdissection PixCell I LCM system available from Arcturus Engineering (Mountainview, Calif.).

Oligonucleotide microarray—In this method oligonucleotide probes capable of specifically hybridizing with the polynucleotides of the present invention are attached to a solid surface (e.g., a glass wafer). Each oligonucleotide probe is of approximately 20-25 nucleic acids in length. To detect the expression pattern of the polynucleotides of the present invention in a specific cell sample (e.g., blood cells), RNA is extracted from the cell sample using methods known in the art (using e.g., a TRIZOL solution, Gibco BRL, USA). Hybridization can take place using either labeled oligonucleotide probes (e.g., 5′-biotinylated probes) or labeled fragments of complementary DNA (cDNA) or RNA (cRNA). Briefly, double stranded cDNA is prepared from the RNA using reverse transcriptase (RT) (e.g., Superscript II RT), DNA ligase and DNA polymerase I, all according to manufacturer's instructions (Invitrogen Life Technologies, Frederick, Md., USA). To prepare labeled cRNA, the double stranded cDNA is subjected to an in vitro transcription reaction in the presence of biotinylated nucleotides using e.g., the BioArray High Yield RNA Transcript Labeling Kit (Enzo, Diagnostics, Affymetix Santa Clara Calif.). For efficient hybridization the labeled cRNA can be fragmented by incubating the RNA in 40 mM Tris Acetate (pH 8.1), 100 mM potassium acetate and 30 mM magnesium acetate for 35 minutes at 94° C. Following hybridization, the microarray is washed and the hybridization signal is scanned using a confocal laser fluorescence scanner which measures fluorescence intensity emitted by the labeled cRNA bound to the probe arrays.

For example, in the Affymetrix microarray (Affymetrix®, Santa Clara, Calif.) each gene on the array is represented by a series of different oligonucleotide probes, of which, each probe pair consists of a perfect match oligonucleotide and a mismatch oligonucleotide. While the perfect match probe has a sequence exactly complimentary to the particular gene, thus enabling the measurement of the level of expression of the particular gene, the mismatch probe differs from the perfect match probe by a single base substitution at the center base position. The hybridization signal is scanned using the Agilent scanner, and the Microarray Suite software subtracts the non-specific signal resulting from the mismatch probe from the signal resulting from the perfect match probe.

Methods of Detecting Expression and/or Activity of Proteins

Expression and/or activity level of proteins expressed in the cells of the cultures of the present invention can be determined using methods known in the arts.

Enzyme linked immunosorbent assay (ELISA): This method involves fixation of a sample (e.g., fixed cells or a proteinaceous solution) containing a protein substrate to a surface such as a well of a microtiter plate. A substrate specific antibody coupled to an enzyme is applied and allowed to bind to the substrate. Presence of the antibody is then detected and quantitated by a colorimetric reaction employing the enzyme coupled to the antibody. Enzymes commonly employed in this method include horseradish peroxidase and alkaline phosphatase. If well calibrated and within the linear range of response, the amount of substrate present in the sample is proportional to the amount of color produced. A substrate standard is generally employed to improve quantitative accuracy.

Western blot: This method involves separation of a substrate from other protein by means of an acrylamide gel followed by transfer of the substrate to a membrane (e.g., nylon or PVDF). Presence of the substrate is then detected by antibodies specific to the substrate, which are in turn detected by antibody binding reagents. Antibody binding reagents may be, for example, protein A, or other antibodies. Antibody binding reagents may be radiolabeled or enzyme linked as described hereinabove. Detection may be by autoradiography, calorimetric reaction or chemiluminescence. This method allows both quantitation of an amount of substrate and determination of its identity by a relative position on the membrane which is indicative of a migration distance in the acrylamide gel during electrophoresis.

Radio-immunoassay (IA): In one version, this method involves precipitation of the desired protein (i.e., the substrate) with a specific antibody and radiolabeled antibody binding protein (e.g., protein A labeled with I¹²⁵) immobilized on a precipitable carrier such as agarose beads. The number of counts in the precipitated pellet is proportional to the amount of substrate.

In an alternate version of the RIA, a labeled substrate and an unlabelled antibody binding protein are employed. A sample containing an unknown amount of substrate is added in varying amounts. The decrease in precipitated counts from the labeled substrate is proportional to the amount of substrate in the added sample.

Fluorescence activated cell sorting (FACS): This method involves detection of a substrate in situ in cells by substrate specific antibodies. The substrate specific antibodies are linked to fluorophores. Detection is by means of a cell sorting machine which reads the wavelength of light emitted from each cell as it passes through a light beam. This method may employ two or more antibodies simultaneously.

Immunohistochemical analysis: This method involves detection of a substrate in situ in fixed cells by substrate specific antibodies. The substrate specific antibodies may be enzyme linked or linked to fluorophores. Detection is by microscopy and subjective or automatic evaluation. If enzyme linked antibodies are employed, a colorimetric reaction may be required. It will be appreciated that immunohistochemistry is often followed by counterstaining of the cell nuclei using for example Hematoxyline or Giemsa stain.

In situ activity assay: According to this method, a chromogenic substrate is applied on the cells containing an active enzyme and the enzyme catalyzes a reaction in which the substrate is decomposed to produce a chromogenic product visible by a light or a fluorescent microscope.

In vitro activity assays: In these methods the activity of a particular enzyme is measured in a protein mixture extracted from the cells. The activity can be measured in a spectrophotometer well using colorimetric methods or can be measured in a non-denaturing acrylamide gel (i.e., activity gel). Following electrophoresis the gel is soaked in a solution containing a substrate and colorimetric reagents. The resulting stained band corresponds to the enzymatic activity of the protein of interest. If well calibrated and within the linear range of response, the amount of enzyme present in the sample is proportional to the amount of color produced. An enzyme standard is generally employed to improve quantitative accuracy.

Ex-vivo detection of radioactive agents generally includes the use of film and/or phoSphoimager screens (e.g. molecular dynamics or Fujifilm BAS 1500). The image of the exposed phosphoimager screen is analyzed using a phosphoimager.

Fluorescent microscopes may be used to image cells labeled with dyes or fluorescent labeled polypeptides (e.g. antibodies) and polynucleotides ex vivo. A wide range of fluorescent microscopes exist which are commercially available.

An ex-vivo technique for analyzing lymph node cells which can also be included in the present invention is the direct use of microscopy to image lymph node cells and their corresponding organelles without the use of a detectable agent. Typically, the microscopy is electron microscopy. This technique has successfully been used for the analysis of mitochondria [e.g. Gunter F. et al., The Journal of Cell Biology, Vol 15, 489-501].

It will be appreciated that more than one of the parameters described hereinabove may be analyzed in order to determine if a lymph node is metastatic. According to a preferred embodiment, the identical cells are analyzed in more than one parameter. According to a preferred embodiment of this aspect of the present invention, cells are analyzed at the individual cell resolution. Preferably small sample sizes (10000 cells) are used. Hence, even sub-populations or rare cells can be distinguished based on their behavior.

An exemplary system for studying more than one parameter in an individual cell is disclosed in U.S. Pat. Appl. No. 20050014201. The Interactive Transparent Individual Cells Biochip Processor (ITICBP) device described therein allows on-line measurement of a vast spectrum of physiological activities of a visually observable individual cell, or a group of cells, using a wide-range of methods such as, morphometry, fluorescence, chromometry, reflectance, electrochemical, and other chemical- and optical-based procedures.

The agents of the present invention (i.e. enzyme substrates or antibodies) may be provided in a kit which, if desired, is presented in a pack which may contain one or more units of the kit of the present invention. The pack may be accompanied by instructions for using the kit. The pack may also be accommodated by a notice associated with the container in a form prescribed by a governmental agency regulating the manufacture, use or sale of laboratory supplements, which notice is reflective of approval by the agency of the form of the compositions. Alternatively or additionally, the agents of the present invention may be provided as articles of manufacture.

As mentioned hereinabove, a subject's lymph node cells may be imaged in vivo and compared to an image of control lymph node cells.

For in vivo use, antibodies are generally either radiolabeled or fluorescent labeled. A variety of methods are known in the art for radiolabeling antibodies [e.g., as described in U.S. Pat. No. 5,985,240; U.S. Pat. No. 4,361,544 and U.S. Pat. No. 4,427,646]. Particular methods of radiolabeling will depend on the radioisotope used.

The amount of an agent used for in in-vivo lymph node cell analysis and the duration of the imaging study will depend upon the label attached to the agent, the body mass of the patient, the nature and severity of the condition being treated, the nature of therapeutic treatments which the patient has undergone, and on the idiosyncratic responses of the patient. For example, one of ordinary skill in the art will recognize that the amount of a radioactive agent used in vivo lymph node cell analysis will depend on the radioisotope used for the labeling of the lymph node cell. Different radioisotopes display different pharmacokinetic properties, such as elimination, clearance from and/or accumulation in biological tissues, and half-life.

Analyzing lymph node cells according to the method of this aspect of the present invention is effected by imaging the agents. In vivo imaging techniques include, but are not limited to radioimaging, fluorescence imaging, biophotonic imaging and magnetic resonance imaging.

The methods described herein for ascertaining lymph node metastasis may be used to select a cancer treatment regimen of a subject. Typically, if a metastatic event is confirmed, a more aggressive treatment regimen will be recommended. For example, in the case of breast cancer, a confirmed lymph node metastasis may result in an axillary lymph node dissection as opposed to a sentinel lymph node dissection. Alternatively or additionally a pharmacological agent may be selected for treating the subject which is appropriate for treating a metastatic event.

As used herein, the phrase “treatment regimen” refers to any active step which may potentially act to prevent some or all of the symptoms associated with cancer. The “treatment regimen” may also potentially alleviate the symptoms or underlying cause of cancer, prolong life expectancy of patients having cancer, as well as potentially aid in complete recovery from a disease.

It will be appreciated that the methods described herein may also be used to simply diagnose cancer. Thus for example, the lymph nodes of a patient close to the site of potential cancerous tissues may be examined. If the lymph nodes are altered in at least one structural or functional parameter with respect to a healthy lymph node cell, this may be used to indicate that the patient has a metastatic cancer. Alternatively, potential cancer cells may be surgically removed and contacted with biopsied autologous lymph node cells. Structural or functional parameters (as described hereinabove) of lymph node cells may be analyzed in order to indicate whether the contacted cells are cancerous or not. Thus, for example if lymph node cells contacted with the potential cancerous cells exhibit a higher MMP activity than lymph node cells which are contacted with identical non-cancerous cells, this would indicate that the cells in question are indeed cancerous. Alternatively or additionally, the lymph node cells of the subject which are contacted with potential cancerous cells may be compared to lymph node cells from a second patient following contact with known tumor cells.

As used herein the term “about” refers to ±10%.

Additional objects, advantages, and novel features of the present invention will become apparent to one ordinarily skilled in the art upon examination of the following examples, which are not intended to be limiting. Additionally, each of the various embodiments and aspects of the present invention as delineated hereinabove and as claimed in the claims section below finds experimental support in the following examples.

### EXAMPLES

Reference is now made to the following examples, which together with the above descriptions, illustrate the invention in a non limiting fashion.

Generally, the nomenclature used herein and the laboratory procedures utilized in the present invention include molecular, biochemical, microbiological and recombinant DNA techniques. Such techniques are thoroughly explained in the literature. See, for example, “Molecular Cloning: A laboratory Manual” Sambrook et al., (1989); “Current Protocols in Molecular Biology” Volumes I-III Ausubel, R. M., ed. (1994); Ausubel et al., “Current Protocols in Molecular Biology”, John Wiley and Sons, Baltimore, Md. (1989); Perbal, “A Practical Guide to Molecular Cloning”, John Wiley & Sons, New York (1988); Watson et al., “Recombinant DNA”, Scientific American Books, New York; Birren et al. (eds) “Genome Analysis: A Laboratory Manual Series”, Vols. 1-4, Cold Spring Harbor Laboratory Press, New York (1998); methodologies as set forth in U.S. Pat. Nos. 4,666,828; 4,683,202; 4,801,531; 5,192,659 and 5,272,057; “Cell Biology: A Laboratory Handbook”, Volumes I-III Cellis, J. E., ed. (1994); “Culture of Animal Cells—A Manual of Basic Technique” by Freshney, Wiley-Liss, N.Y. (1994), Third Edition; “Current Protocols in Immunology” Volumes I-III Coligan J. E., ed. (1994); Stites et al. (eds), “Basic and Clinical Immunology” (8th Edition), Appleton & Lange, Norwalk, Conn. (1994); Mishell and Shiigi (eds), “Selected Methods in Cellular Immunology”, W. H. Freeman and Co., New York (1980); available immunoassays are extensively described in the patent and scientific literature, see, for example, U.S. Pat. Nos. 3,791,932; 3,839,153; 3,850,752; 3,850,578; 3,853,987; 3,867,517; 3,879,262; 3,901,654; 3,935,074; 3,984,533; 3,996,345; 4,034,074; 4,098,876; 4,879,219; 5,011,771 and 5,281,521; “Oligonucleotide Synthesis” Gait, M. J., ed. (1984); “Nucleic Acid Hybridization” Hames, B. D., and Higgins S. J., eds. (1985); “Transcription and Translation” Hames, B. D., and Higgins S. J., eds. (1984); “Animal Cell Culture” Freshney, R. I., ed. (1986); “Immobilized Cells and Enzymes” IRL Press, (1986); “A Practical Guide to Molecular Cloning” Perbal, B., (1984) and “Methods in Enzymology” Vol. 1-317, Academic Press; “PCR Protocols: A Guide To Methods And Applications”, Academic Press, San Diego, Calif. (1990); Marshak et al., “Strategies for Protein Purification and Characterization—A Laboratory Course Manual” CSHL Press (1996); all of which are incorporated by reference as if fully set forth herein. Other general references are provided throughout this document. The procedures therein are believed to be well known in the art and are provided for the convenience of the reader. All the information contained therein is incorporated herein by reference.

### Example 1

### Analysis of Cell Morphology in Tumor-Free or Metastatic Lymph Nodes

Lymph nodes comprise a number of cell types, including lymphocytes, dendritic cells (DC), macrophages and blast cells. The following experiment was performed to analyze the size of specific cell populations in metastasized and non-metastasized lymph nodes using digital image analysis.

Materials and Methods

Tissue Samples: Tumor tissue samples and lymph nodes cells from 35 breast cancer patients were obtained from surgical departments of “Assaf Harofeh” Medical Center, Zerifin 70300, Israel. Altogether, 50 lymph nodes were analyzed. Twelve investigated patients had metastases in axillary lymph nodes and 23 were lymph node negative. In four out of twelve node-positive patients, cells from metastatic and tumor-free lymph nodes were studied. The research was conducted in accordance with the principles of the Declaration of Helsinki. The pathological tumor tissue examinations were carried out by the pathology departments of the Medical Center. The tumor tissue samples were stored in liquid nitrogen until use.

Lymph node cell collection: The lymph node cells were extracted by pathologists following lymph node (sentinel and/or axillary) removal from breast tissue, and washed in RPMA-1640 medium in order to remove excessive tissue residue. Each lymph node was cut into two lobes and its internal side was scanned using a Luer syringe filled with RPMA-1640 medium. During the scan, a constant pressure applied to the syringe piston produced a steady stream of fluid that hit the tissue and extracted cells from the lymph node. The cell-loaded fluid flowed over the lobe's “surface” and was collected in a Petri dish.

Protocol for lymph node cell preparation: Lymph node cell suspension collected as described above, was centrifuged (5 minutes, 1000 rpm) at room temperature. Erythrocyte lysis was carried out by treating the pellet for 20 seconds with two volumes of cold distilled water, followed by addition of one volume of 3.5% NaCl. Cells were washed again and maintained in RPMI-1640 medium, supplemented with 10% heat-inactivated fetal calf serum, 100 U/ml penicillin, 100 μg/ml streptomycin, 2% glutamine, 2% sodium pyruvate and 2% HEPES (Biological Industries, Kibbutz Beit Haemek, Israel) at a final concentration of 2×10⁶ cells/ml.

Viability, which was determined by PI exclusion test, was always higher than 90%. Residual cell preparations, in which the number of dead cells exceeded 10%, were excluded. The number of cells obtained from a single node was in the range of 0.2×10⁶ cells to 15×10⁶ cells.

Protocol for breast tumor dissociation: A small piece of intact un-fractionized tumor tissue was derived from each breast cancer patient and delivered to the Pathology laboratory together with the LN cells. The tumor tissue was washed three times in PBS and then cut into several small fractions (<1 mm³) according to the number of the lymph nodes that were delivered from the same patient.

Protocol for autologous tumor stimulation: Freshly isolated lymph node (LN) cells were suspended in complete RPMI medium at 2×10⁶ cells/ml. Cells were incubated in the presence or absence of a small piece (<1 mm³) of intact un-fractionated autologous tumor tissue for 18 hours at 37° C. in the presence of 5% CO₂.

Size measurements of specific cell populations: The size of the specific cell populations was measured using Image analysis. Olympus IX81 epi-fluorescence inverted microscope, was equipped with 100 W Hg excitation lamp, with a sub-micron SCAN-IM motorized stage (Marzhauser, Wetzlar-Steindorf, Germany), and a cooled highly sensitive ORCA II C4742-98 camera (Hamamatsu, Hamamatsu-City, Japan) providing 14-bit digitization. Software: MetaMorph (MM—from Molecular Devices Corporation, Sunnyvale, Calif.) was used for controlling the camera, the microscope (Objectives, filters and shutters), microscope stage, as well as for on-/off-line image/signal analysis.

Results

As illustrated in FIG. 1A, a significant difference between the cell size of tumor-free and metastatic lymph nodes was observed, the latter exhibiting a larger image area (p<0.0001). In addition, metastatic lymph nodes exhibited a greater increase in cell area following autologous tumor tissue (TT) activation than tumor-free lymph nodes (FIG. 1B; p<0.0001).

### Example 2

### Monitoring Intracellular Enzymatic Reaction Rates in Individual Live Lymph Node Cells

In order to ascertain whether enzymatic reaction rates were different in metastatic lymph nodes and non-metastatic lymph nodes, the intracellular esterase activity in resting and tumor activated individual cells from lymph nodes of cancer patients was measured by monitoring the hydrolysis of a fluoresceindiacetate (FDA) substrate.

Materials and Methods

Lymph and tumor cell collection and preparation were performed as described in Example 1 hereinabove. Approximately 600 cells per lymph node were incubated with 1.2 μM FDA substrate (commercially available from Riedle—de Haen (Seelze, Germany) Tumor tissue activated and non-activated lymph node cells were examined. Tumor tissue activation was performed as described in Example 1 hereinabove.

Measurement of FDA hydrolysis rate: The kinetics of the biochemical reactions for individual cells was calculated following repeated periodic measurements of the same cells. Six emissions were collected approximately at 30 second intervals and the slopes were calculated from “fluorescence intensity” (FI) versus “time” graphs. Damaged and dead cells, which did not show an increase in FI upon exposure to FDA, were excluded from the image analysis. The enzymatic hydrolysis of FDA and the intracellular accumulation of fluorescein by individual cells derived from lymph nodes were measured using an Image system. Images were acquired by a motorized upright epi-fluorescence Olympus BX51 microscope, equipped with motorized polarization filters for fluorescence polarization measurements. Cells were illuminated by a Mercury light source. The emitted fluorescence was imaged using a CoolSNAP HQ monochrome CCD camera. Digital image analysis of cellular fluorescence was performed by Image Pro plus software.

Mathematical calculations and statistical analysis: Data obtained by digital image analysis was used to calculate fluorescein accumulation rate. All values are presented as average ±s.e.m. The comparisons between groups were performed by the t-test, ANOVA, MANOVA or some non-parametric tests (Wilcoxon, Kruskal-Wallis, and van der Waerden) for the small groups.

Results

A sharp increase in FDA hydrolysis rate was demonstrated in lymphocytes derived from metastatic lymph nodes (1.440±0.023 in control cells and 2.368±0.043 in tumor tissue incubated cells, P<0.0001), while a decrease of this parameter was exhibited in the lymphocytes that were evoked from tumor-free lymph nodes (1.04±0.028 in control group and 0.473±0.024 in tumor tissue incubated cells, P<0.0001).

As illustrated in FIGS. 2A-B and FIG. 3, the percent of cells in individual lymph nodes with higher FDA slopes (>1.5) doubled in metastatic LNs after autologous TT activation, in contrast to a small decrease in tumor free LNs. This difference was statistically significant according to some non-parametric tests (Wilcoxon, Kruskal-Wallis, van der Waerden).

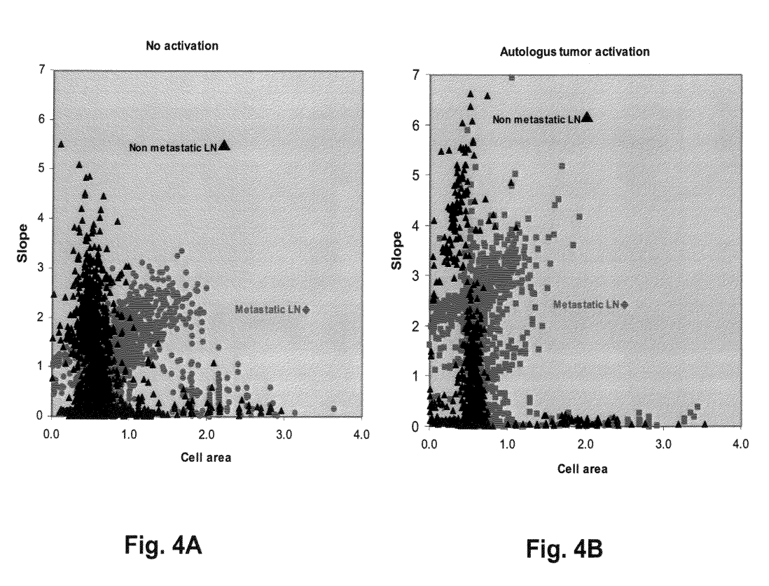

Since the evaluation of the studied parameter was performed by digital image analysis, specific cell populations were investigated for their ability to hydrolyze FDA. The correlation between functional and morphological characteristics of individual LN cells was analyzed prior to and following autologous tumor tissue activation. FIGS. 4A-B are scatter plots of the intracellular reaction rates vs. cell area of individual cells derived from tumor free or metastatic lymph nodes, before and after autologous tumor activation. The figures clearly show that based on these parameters, cells from tumor invaded nodes can easily be discriminated from cells derived from tumor free ones. Moreover, in tumor free nodes, the increase in reaction rate following autologous tumor cell activation is limited to a small size cell—subset, while in invaded lymph nodes a significant increase in reaction rate is evident in all tested cells, which is accompanied by a small decrease in average cell size.

### Example 3

### Analysis of Early Lymphocyte Activation Markers on Individual Lymph-Node Cells

Since the difference in the ability of lymph node cells to utilize FDA may be a consequence of the various activation states of these cells, early activation surface marker (CD71) expression was analyzed for the individual lymphocytes.

Materials and Methods

Following the kinetic measurements described in Example 2 hereinabove, cells were fixed on live cell array with formaldehyde (4%) for 10 minutes, washed with PBS and stained with anti-human phycoerythrin (PE) conjugated antibodies for CD71 or CD69 receptors in optimal working dilution for 30 minutes at RT. (Anti-human CD71R-PE antibodies were purchased from Chemicon Europe, U.K., and anti-human CD69 PE-conjugated antibodies were obtained from Bioscience). Following incubation, lymph node cells were washed and measured by Olympus motorized upright epi-fluorescence BX51 microscope, equipped with a Mercury light source. The emitted fluorescence was imaged using a CoolSNAP HQ monochrome CCD camera, or DVC color camera. Digital image analysis from cellular fluorescence was performed by Image Pro plus software.

Results

As illustrated in FIG. 5B, cells derived from metastatic lymph nodes exhibited a relatively homogeneous CD71 expression on their cell surface. An increase in heterogeneity was observed following activation with autologous tumor tissue. Cells, derived from tumor-free lymph node displayed a less homogeneous CD71 expression which decreased even more following activation (FIG. 5A).

A correlation was observed between the cell's ability to hydrolyze FDA and expression of early lymphocyte activation marker (CD71) on its cell surface. Thus, as illustrated in FIG. 6, cells with higher CD71 expression exhibited lower FDA reaction rates. This effect was more pronounced following autologous tumor tissue activation. The FDA reaction rate increased significantly following autologous tumor tissue activation for cells with both high and low CD71 expression.

### Example 4

### Analysis of Matrix Metalloproteinases (MMPs) in Individual Lymph-Node Cells

The matrix metalloproteinases activity of lymph node cells was measured following incubation in the presence of either autologous tumor, PMA (10 ng/mL) or in complete medium, without any stimulant.

Materials and Methods

DQ Gelatin FITC conjugated substrate (EnzChek; Molecular probes, IS) (5 μg/mL) dissolved in liquid low-melt agarose (0.5%) was mixed with live LN cell suspension (final concentration of 4×10⁶ cells/mL). To a number of wells of a 96-well microtiter plate, 30 μl of this mixture was added. The plate was subsequently centrifuged so as to cause dispensed cells to settle at the bottom of the wells and loaded onto the LiveCell™ array. Following cell settling, the microtiter plate was cooled down to 4° C. for 5 minutes to enable gelling. The cells were covered with complete medium and incubated at 37° C. in the presence of 5% CO₂. Since the substrate is efficiently digested by MMP-2 (Gelatinase A) and MMP-9 (Gelatinase B) to yield highly fluorescent peptides, MMP activity of LN cells was measured by measuring the amount of enzymatically cleaved fluorescent peptides. Thus, single cell MMP activity was visualized as a fluorescent signal at the location of the MMP secreting cell.

The cells were imaged using an Olympus motorized inverted microscope IX81 equipped with a Mercury light source. The emitted fluorescence was imaged using CoolSNAP HQ monochrome CCD camera. Digital image analysis from cellular fluorescence was performed by Image Pro plus software.

Results

As illustrated in FIGS. 7A-B, MMP activity (Mean Intensity measured 2 h and 24 h culture within LiveCell array) was observed in individual cells originating from tumor free or metastatic LNs with or without TT activation. Cells derived from metastatic lymph nodes exhibited higher MMP activity both prior to and following activation with autologous tumor tissue.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims. All publications, patents and patent applications mentioned in this specification are herein incorporated in their entirety by reference into the specification, to the same extent as if each individual publication, patent or patent application was specifically and individually indicated to be incorporated herein by reference. In addition, citation or identification of any reference in this application shall not be construed as an admission that such reference is available as prior art to the present invention.

What is claimed is:

1. A method of determining lymph node metastasis in a subject, the method comprising determining at least one structural or functional parameter of a lymph node cell of the subject, wherein an alteration in said at least one structural or functional parameter with respect to a healthy lymph node cell is indicative of lymph node metastasis.

2. The method of claim 1, wherein said lymph node cell is a lymphocyte.

3. The method of claim 1, wherein said lymph node cell is viable.

4. The method of claim 1, wherein said lymph node cell is non-malignant.

5. The method of claim 1, wherein said lymph node cell is isolated.

6. The method of claim 1, wherein said structural parameter of said lymph node cell is a cell size, an organelle size, a cell number and an organelle number.

7. The method of claim 1, wherein said functional parameter of said cell is at least one of the following:
i. an expression level or activity of an enzyme;
ii an expression level or activity of a cell surface marker;
iii. an expression level or activity of a chemokine receptor;
iv. an expression level or activity of a of a chemokines; and/or
v. a proliferation rate.

8. The method of claim 7, wherein said enzymatic activity is an esterase activity or a metalloproteinase activity.

9. The method of claim 1 further comprising contacting said lymph node cell of the subject and/or said healthy lymph node cell with a tumor cell prior to determining said at least one structural or functional parameter.

10. The method of claim 1, wherein said lymph node is a breast lymph node.

11. A method of determining a cancer treatment regimen of a subject comprising;
(a) determining at least one structural or functional parameter of a lymph node cell of the subject; and
(b) selecting a treatment according to the results of (a), thereby determining a cancer treatment regimen.

12. The method of claim 11, wherein said lymph node cell is a lymphocyte.

13. The method of claim 11, wherein said lymph node cell is non-malignant.

14. The method of claim 11, wherein said functional parameter of a lymph node cell is at least one of the following:
i. an expression level or activity of an enzyme;
ii an expression level or activity of a cell surface marker;
iii. an expression level or activity of a chemokine receptor;
iv. an expression level or activity of a of a chemokines; and/or
v. a proliferation rate.

15. The method of claim 14, wherein said enzymatic activity is an esterase activity or a metalloproteinase activity.

16. The method of claim 11, further comprising contacting said lymph node cell and/or said healthy lymph node cell with a tumor cell prior to determining said at least one structural or functional parameter.

17. The method of claim 11, wherein said lymph node is a breast lymph node.

18. A kit for determining lymph node metastasis in a subject, the kit comprising a packaging material which comprises at least one agent for determining a structural or functional parameter of a lymph node cell of the subject.

19. The kit of claim 18, wherein said at least one agent for determining a functional parameter of said lymph node cell is a substrate for an enzyme.

20. The kit of claim 18, wherein said at least one agent for determining a functional parameter of said cell is an antibody against a cell surface marker.

21. An article of manufacture comprising packaging material and an agent identified for determining lymph node metastasis in a subject being contained within said packaging material, said agent being capable of determining a structural or functional parameter of a lymph node cell of the subject.

22. The article of manufacture of claim 21, wherein said at least one agent for determining a functional parameter of said lymph node cell is a substrate for an enzyme.

23. The article of manufacture of claim 22, wherein said at least one agent for determining a functional parameter of said lymph node cell is an antibody against a cell surface marker.

24. A method of diagnosing cancer in a subject, the method comprising determining at least one structural or functional parameter of a lymph node cell of the subject, wherein an alteration in said at least one structural or functional parameter with respect to a healthy lymph node cell is indicative of cancer, thereby diagnosing cancer in a subject.

25. The method of claim 24, further comprising contacting cells of interest with said lymph node cell prior to said determining.
